(19) United States
(12) Patent Application Publication
(10) Pub. No.: US 2017/0079572 A1
(43) Pub. Date: Mar. 23, 2017

(54) METHOD AND APPARATUS FOR EVALUATING EXERCISE CAPACITY

(71) Applicant: Samsung Electronics Co., Ltd., Suwon-si (KR)

(72) Inventors: Dae-Geun JANG, Yongin-si (KR); Byunghoon KO, Hwaseong-si (KR); SangKon BAE, Seongnam-si (KR)

(73) Assignee: Samsung Electronics Co., Ltd., Suwon-si (KR)

(21) Appl. No.: 15/070,631

(22) Filed: Mar. 15, 2016

(30) Foreign Application Priority Data
Sep. 23, 2015 (KR) 10-2015-0134630

(51) Int. Cl. A61B 5/22; A61B 5/00; A63B 24/00; A61B 5/024

(52) U.S. Cl. CPC A61B 5/222; A61B 5/02438; A61B 5/7278; A61B 5/7275; A61B 5/4866; A63B 24/0062; A63B 24/0075; A63B 2024/0065

(57) ABSTRACT

A method and apparatus for evaluating exercise capacity are provided. The method of evaluating exercise capacity involves obtaining heart rate distribution data based on measured heart rate of a user, calculating heart rate statistics based on the heart rate distribution data, and evaluating an exercise capacity of the user based on the heart rate statistics.

### CROSS-REFERENCE TO RELATED APPLICATION(S)

This application claims the benefit under 35 USC 119(a) of Korean Patent Application No. 10-2015-0134630, filed on Sep. 23, 2015, in the Korean Intellectual Property Office, the entire disclosure of which is incorporated herein by reference for all purposes.

### BACKGROUND

1. Field

The following description relates to a method and apparatus for evaluating exercise capacity based on heart rate statistics.

2. Description of Related Art

A physician may evaluate cardio-respiratory fitness of a patient, using various measuring devices including a gas analyzer and a blood glucose meter. However, the general public lacks the technical expertise and medical knowledge to operate these measuring devices to conduct exercise stress tests. Thus, the general public are unable to readily use such measuring devices to determine their cardio-respiratory fitness. Accordingly, various methods have been proposed to conveniently measure a physical fitness level or exercise capacity in daily life.

When a physician assesses a patient's physical fitness by monitoring his or her heart rate, the physical fitness level and exercise capacity of the patient may be evaluated based on an assumption of a linear relationship between the patient's heart rate and the exercise intensity. However, there may be a nonlinear relationship between an increase in the exercise intensity and the metabolic demand. In such a case, when the exercise intensity increases, a large error may occur in a calculated value of a metabolic characteristic that is estimated based on the heart rate. Although various non-metabolic indices such as the age, gender, and weight of a user, for example, may be used to supplement the nonlinear relationship, the physiological basis and accuracy in measurement results may still be limited because the indices are not information directly indicating metabolic characteristics of a user.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a method of evaluating exercise capacity involves obtaining heart rate distribution data based on measured heart rate of a user, calculating heart rate statistics based on the heart rate distribution data, and evaluating an exercise capacity of the user based on the heart rate statistics.

The calculating of the heart rate statistics may involve calculating at least one of skewness, kurtosis, and quantile of the heart rate distribution data.

The evaluating of the exercise capacity may involve estimating at least one of a maximum oxygen uptake, a ventilatory threshold, a lactate threshold, and a maximal heart rate based on the heart rate statistics, and evaluating the exercise capacity of the user based on a result of the estimating.

The estimating of the exercise capacity may involve applying the heart rate statistics to an estimation regression equation to estimate at least one of the maximum oxygen uptake, the ventilatory threshold, the lactate threshold, and the maximal heart rate of the user.

The general aspect of the method may further involve receiving body information of the user, and the evaluating of the exercise capacity may involve evaluating the exercise capacity based on the body information and the heart rate statistics.

The body information of the user may include at least one of a gender, an age, a height, a weight, a waist-hip ratio (WHR), and a body mass index (BMI) of the user.

The general aspect of the method may further involve measuring the heart rate.

The measuring may further involve measuring a heart rate of the user while the user is performing an exercise in which a workload increases.

The general aspect of the method may further involve selecting an exercise program based on the exercise capacity of the user from pre-stored exercise programs, receiving a feedback on an exercise performance result from the user performing an exercise based on the selected exercise program, and adjusting the exercise program based on information associated with the received feedback.

The general aspect of the method may further involve comparing the exercise capacity of the user to a standard exercise capacity based on body information of the user; and providing information on an exercise based on a physical fitness level of the user based on a result of the comparing.

The general aspect of the method may further involve predicting a metabolic disease risk of the user based on the exercise capacity, and providing a warning about the metabolic disease risk.

The predicting may involve calculating a healthiness grade of the user based on the exercise capacity of the user, and estimating a risk of death based on the healthiness grade of the user.

The general aspect of the method may further involve providing a lifestyle prescription to reduce the metabolic disease risk of the user.

In another general aspect, a non-transitory computer readable medium storing instructions that cause a processor to perform the general aspect of the method described above is provided.

In yet another general aspect, an apparatus for evaluating exercise capacity includes a measurer configured to measure a heart rate of a user, and a processor configured to calculate heart rate statistics from heart rate distribution data obtained based on the measured heart rate and evaluate an exercise capacity of the user based on the heart rate statistics.

The general aspect of the apparatus may further include a memory configured to store at least one of the heart rate, the heart rate distribution data, and the heart rate statistics.

The processor may be configured to calculate at least one of skewness, kurtosis, and quantile of the heart rate distribution data.

The processor may be configured to estimate at least one of a maximum oxygen uptake, a ventilatory threshold, a lactate threshold, and a maximal heart rate by applying the heart rate statistics to an estimation regression equation, and evaluate the exercise capacity of the user based on a result of the estimating.

The general aspect of the apparatus may further include a receiver configured to receive body information of the user, and the processor may be configured to evaluate the exercise capacity of the user based on the body information and the heart rate statistics.

The measurer may be configured to measure a heart rate of the user while the user is performing an exercise in which a workload increases.

In yet another general aspect, an apparatus for evaluating exercise capacity includes a processor configured to obtain heart rate distribution data based on a heart rate of a person measured while performing a predetermined exercise regime, and evaluate an exercise capacity of the person based on a calculation performed on the heart rate distribution data, and the apparatus is a wearable device or a mobile device.

The general aspect of the apparatus may further include a heart rate sensor configured to measure the heart rate of the person.

The calculation performed on the heart rate distribution data may involve determining at least one of a maximum oxygen uptake, a ventilatory threshold, a lactate threshold, and a maximal heart rate.

Other features and aspects will be apparent from the following detailed description, the drawings, and the claims.

### BRIEF DESCRIPTION OF THE DRAWINGS

FIG. 1 illustrates an example of a graph that may be used to evaluate exercise capacity of a person based on his or her heart rate.

FIG. 2 is a flowchart illustrating an example of a method of evaluating exercise capacity.

FIG. 3 illustrates an example of a heart rate distribution data.

FIG. 4 is a flowchart illustrating another example of a method of evaluating exercise capacity.

FIG. 5 is a flowchart illustrating still another example of a method of evaluating exercise capacity.

FIG. 6 is a block diagram illustrating an example of an exercise capacity evaluation apparatus.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent to one of ordinary skill in the art. The sequences of operations described herein are merely examples, and are not limited to those set forth herein, but may be changed as will be apparent to one of ordinary skill in the art, with the exception of operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

The following specific structural or functional descriptions are exemplary to merely describe the examples, and the scope of the examples is not limited to the descriptions provided in the present specification.

It will be understood that, although the terms first, second, etc. may be used herein to describe various elements, these elements should not be limited by these terms. These terms are only used to distinguish one element from another. For example, a first signal could be termed a second signal, and, similarly, a second signal could be termed a first signal without departing from the teachings of the disclosure.

It will be understood that when an element or layer is referred to as being “on”, “attached to”, or “connected to” another element or layer, it can be directly on or connected to the other element or layer or through intervening elements or layers may be present. In contrast, when an element is referred to as being “directly on”, “directly attached to”, or “directly connected to” another element or layer, there are no intervening elements or layers present. Other words used to describe the relationship between elements or layers should be interpreted in a like fashion (e.g., “between” versus “directly between,” “adjacent” versus “directly adjacent,” “on” versus “directly on”).

The terminology used herein is for the purpose of describing particular examples only and is not to limit the examples. As used herein, the singular forms “a”, “an”, and “the” are intended to include the plural forms as well, unless the context clearly indicates otherwise. It will be further understood that the terms “include/comprise” and/or “have” when used in this specification, specify the presence of stated features, integers, steps, operations, elements, components, and/or combinations thereof, but do not preclude the presence or addition of one or more other features, numbers, steps, operations, elements, components, and/or groups thereof.

Unless otherwise defined, all terms including technical and scientific terms used herein have the same meaning as commonly understood by one of ordinary skill in the art to which examples belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

The following example embodiments may be applied to evaluate exercise capacity of a user. Example embodiments may be implemented to be various forms, for example, a personal computer, a laptop computer, a tablet computer, a smartphone, a television, a smart appliance, an intelligent vehicle, and a wearable device. Example embodiments may be applied to providing an exercise program suitable for a user or informing the user of a metabolic disease risk by evaluating exercise capacity of the user based on a heart rate measured from the user using, for example, a smartphone, a mobile device, and a smart home system. Example embodiments may also be applied to, for example, a healthcare service for the user. Hereinafter, reference will be made in detail to examples with reference to the accompanying drawings, wherein like reference numerals refer to like elements throughout.

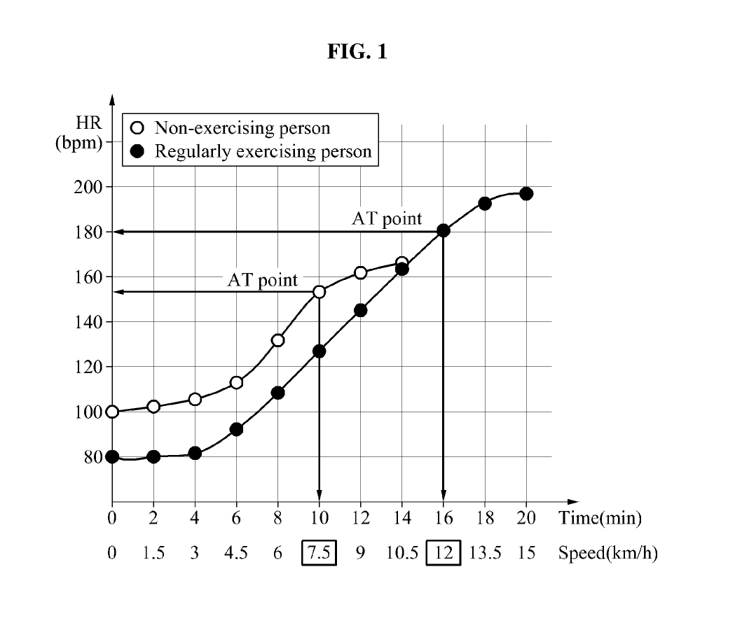

FIG. 1 illustrates an example of a graph for illustrating a principle used to evaluate exercise capability based on heart rate statistics. FIG. 1 illustrates heart rate changes in a user who regularly exercises and heart rate changes in a user who does not regularly exercise while performing a graded load exercise. Hereinafter, the user who regularly performs an exercise is also referred to as a regularly exercising person or a physically fit person, and the user who does not exercise regularly is referred to as a non-exercising person or an under-exercising person. In a graph of FIG. 1, a heart rate of the regularly exercising person is indicated by a dot, •, and a heart rate of the non-exercising person is indicated by an empty dot, ∘.

Referring to FIG. 1, the heart rate of a person increases proportionally to the exercise intensity, but the rate of increase in heart rate radically decreases after reaching an anaerobic threshold (AT) point. The anaerobic threshold (AT) point refers to an exercise intensity in which the blood concentration of lactate arises rapidly due to change in metabolism. The oxygen consumption of a muscle under exercise, for example, increases in proportion to an increase in the intensity of the exercise being performed. In this disclosure, the terms “intensity of exercise” and “exercise intensity” are interchangeably used with the term “workload”. In this example, an energy supply is provided to the muscle through an anaerobic metabolic process as well as through an aerobic metabolic process from a predetermined exercise intensity. As a result of anaerobic metabolism, lactic acid starts accumulating in the tissue and blood stream and an output of carbon dioxide increases. Also, to emit the increased carbon dioxide, an amount of body ventilation excessively increases. An exercise intensity or oxygen consumption corresponding to a point at which such phenomenon starts is also referred to as an anaerobic threshold. The anaerobic threshold includes, for example, a lactate threshold and a ventilatory threshold. The lactate threshold indicates a point at which a lactate density radically increases in blood with respect to an increase in the workload in a relationship between the exercise intensity and the lactate density.

In most people, the anaerobic threshold is estimated to be about 50 to 60% of a maximum exercise intensity. In a case of a long distance runner, the anaerobic threshold increases to be about 80% of the maximum exercise intensity. As such, a heart rate of a person at the anaerobic threshold has a meaningful relationship with, for example, the lactate threshold and a maximum oxygen uptake, and provides information regarding the exercise capacity and physical strength of the person.

Referring to FIG. 1, a regularly exercising person or a physically fit person has a stable-state heart rate, or a resting heart rate, that is lower than that of a non-exercising person. Also, the heart rate of the regularly exercising person increases more slowly during a graded load exercise in comparison to the heart rate of the non-exercising person.

In this example, the regularly exercising person reaches the lactate threshold at an exercise intensity 12, and the non-exercising person reaches the lactate threshold at an exercise intensity 7.5. Since the regularly exercising person reaches the lactate threshold or the ventilatory threshold at an exercise intensity higher than that of the non-exercising person, the regularly exercising person may reach the maximal heart rate and the maximum oxygen uptake at the higher exercise intensity when compared to the non-exercising person. Such different characteristics in heart rate trend between the regularly exercising person and the non-exercising person may be expressed by heart rate statistics as well as a heart rate trend as represented by a graph of FIG. 1.

The regularly exercising person may have the lower resting heart rate having a minimal heart rate lower than that of the non-exercising person. Also, during the graded load exercise, the regularly exercising person may have the higher maximal heart rate and longer exercise duration when compared to the non-exercising person.

Since the regularly exercising person has the higher maximal heart rate during the graded load exercise, the regularly exercising person may have a greater physical strength and a higher exercise level when compared to the non-exercising person.

Also, due to the relatively slow rate of increase in heart rate and a relatively high physical fitness level achieved before reaching the AT point, the heart rate distribution data of the regularly exercising person may exhibit a lower skewness and a higher kurtosis during the graded load exercise in comparison to the heart rate distribution data of the non-exercising person.

In an example, a physical strength and exercise capacity expressed by, for example, a maximum oxygen uptake, a lactate threshold, a ventilatory threshold, and a maximal heart rate may be evaluated based on heart rate statistics measured during a graded load exercise in consideration of the aforementioned theory.

In an example, an exercise capacity of a user may be evaluated based on heart rate statistics to minimize an error due to noise components included in heart rate information, thereby enhancing accuracy of the evaluating. Also, the exercise capacity may be evaluated based on the heart rate statistics having a relatively small amount of calculations so as to be applied to low power devices, for example, a mobile device and a wearable device.

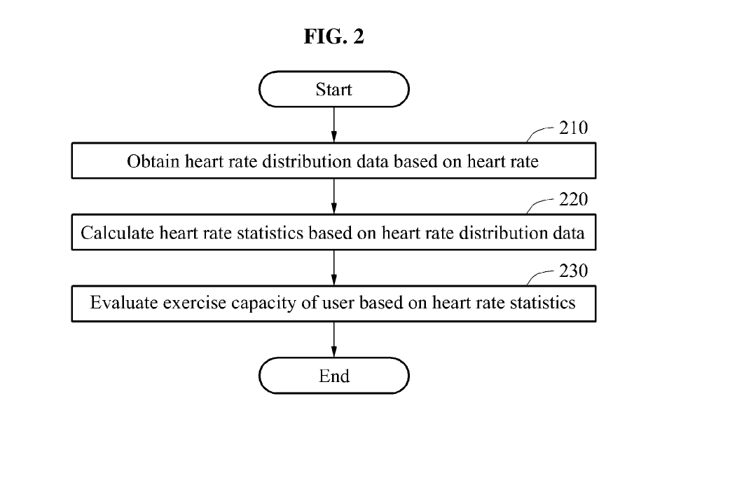

FIG. 2 is a flowchart illustrating an example of a method of evaluating exercise capacity.

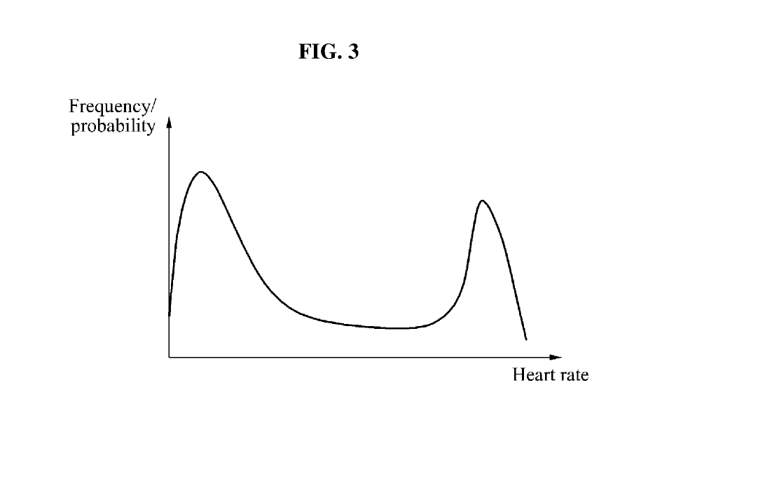

Referring to FIG. 2, in operation 210, an apparatus for evaluating an exercising capacity generates a heart rate distribution data based on a measured heart rate of a user. Descriptions related to an example of the heart rate distribution data generated by the apparatus will be provided with reference to FIG. 3. In FIG. 3, the heart rate distribution data is graphically illustrated as a heart rate distribution curve. However, in another example, the heart rate distribution data may be embodied in various forms, including graphical and non-graphical forms, matrixes, data structures and the like.

In operation 220, the apparatus calculates heart rate statistics based on the heart rate distribution data generated in operation 210. Based on the heart rate distribution data, the apparatus calculates at least one of heart rate statistics such as quantile, kurtosis, and skewness of the heart rate, standard deviation, a most frequent value, for example, a mode value, a median value, and a mean value of the heart rate, and an intermediate value between a maximal heart rate and a minimal heart rate. The quantile is understood as, for example, a value of a point corresponding to 25% or 75% of overall distributed heart rates in the heart rate distribution data. The apparatus calculates the heart rate statistics based on a heart rate signal averaged at a predetermined time interval, for example, one minute, in the heart rate distribution data.

The apparatus calculates the skewness and the kurtosis of the heart rate from the heart rate distribution data based on Equations 1 and 2 as shown below.

$$\begin{matrix}Skewness = E [ {( \frac{X - μ}{σ} )}^{3} ] & [ Equation 1 ] \\ Kurtosis = E [ {( \frac{X - μ}{σ} )}^{4} ] & [ Equation 2 ]\end{matrix}$$

In Equations 1 and 2, μ denotes a mean value, a denotes a standard deviation, and E denotes an expectation operator.

The apparatus sorts heart rate data by applying a sorting algorithm to the heart rate distribution data and calculates the quantile by selecting a predetermined distribution range, for example, a heart rate value corresponding to a level of about 25%. The sorting algorithm includes, for example, a selection sort, a bubble sort, an insertion sort, a merge sort, a quick sort, and a heap sort.

In operation 230, the apparatus evaluates exercise capacity of the user based on the heart rate statistics. The apparatus estimates at least one of the maximal heart rate, a lactate threshold, a ventilatory threshold, and a maximum oxygen uptake of the user based on the heart rate statistics, and evaluates the exercise capacity of the user based on a result of the estimating.

FIG. 3 illustrates an example of a heart rate distribution data. Referring to FIG. 3, an apparatus for evaluating exercise capacity generates a heart rate distribution data, for example, in a form of histogram. In the heart rate distribution data of FIG. 3, a horizontal axis represents a heart rate and a vertical axis represents a distribution probability or a frequency.

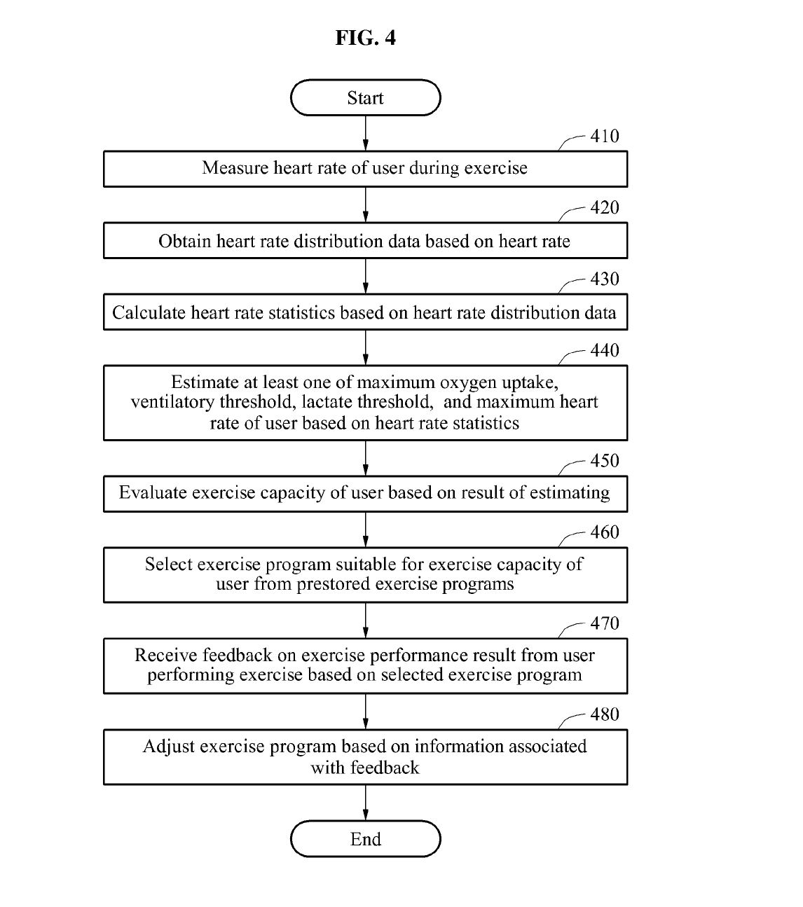

FIG. 4 is a flowchart illustrating another example of a method of evaluating exercise capacity.

Referring to FIG. 4, in operation 410, an apparatus for evaluating exercise capacity measures a heart rate of a user while the user is performing an exercise. For example, the apparatus measures a heart rate sensed from the user while the user is performing a graded load exercise on equipment or while the user is performing a daily exercise in which an exercise load increases. Examples of equipment include a treadmill, a bicycle ergometer, a bench step and the like. Examples of daily exercises in which the exercise load increases include running, jogging, walking, step climbing and the like.

According to one example, the apparatus is implemented as a wearable device including a heart rate sensing device or a heart rate system provided in a diversified form. For example, the wearable device may be a watch type wearable device, a bracelet type wearable device, a chest type wearable device, an in-ear type wearable device, and the like. According to another example, the apparatus is implemented as a mobile device connected with the wearable device through a wired or wireless communication. The heart rate sensing device may include, for example, a photoplethysmogram (PPG) sensor. The apparatus may measure a heart rate of a user using various types of heart rate sensing devices or heart rate systems.

In operation 420, the apparatus generates a heart rate distribution data based on the heart rate measured in operation 410.

In operation 430, the apparatus calculates heart rate statistics based on the heart rate distribution data. As an example, the apparatus calculates heart rate statistics with respect to all heart rate data expressed as the heart rate distribution data. Thus, the apparatus does not need to perform a preprocessing for feature point extraction, and minimizes an influence of noise occurring during an exercise.

In operation 440, the apparatus estimates at least one of a maximum oxygen uptake, a ventilatory threshold, a lactate threshold, and a maximal heart rate of the user based on the heart rate statistics calculated in operation 430. As an example, the apparatus applies the heart rate statistics to a linear estimation regression equation to estimate a metabolic index, for example, the maximum oxygen uptake, the ventilatory threshold, the lactate threshold, and the maximal heart rate of the user.

The apparatus applies the heart rate statistics to a first estimation regression equation, for example, Y=α×X+β, to estimate the metabolic index, for example, the maximum oxygen uptake, the ventilatory threshold, the lactate threshold, and the maximal heart rate of the user. In this example, a value applied to X of the first estimation regression equation is a value of the heart rate statistics.

In the estimation regression equation, a most frequent value, for example, a mode value may be applied as the heart rate statistics, X as well as skewness, kurtosis, and quantile. In this example, coefficients α and β of the estimation regression equation may be differently determined based on the heart rate statistics and a metabolic index, Y. As an example, the coefficients α and β determined when the metabolic index to be estimated is the maximum oxygen uptake differ from the coefficients α and β determined when the metabolic index to be estimated is the maximal heart rate.

In operation 450, the apparatus evaluates an exercise capacity of the user based on a result of the estimating in operation 440, for example, the estimated metabolic index of the user. As an example, when the estimated maximum oxygen uptake exceeds a predetermined reference, the apparatus evaluates that the exercise capacity of the user is relatively high. As another example, when the estimated maximal heart rate is lower than a predetermined reference, the apparatus evaluates that the exercise capacity of the user is relatively low.

In operation 460, the apparatus selects an exercise program suitable for the exercise capacity evaluated in operation 450 from pre-stored exercise programs. Also, the apparatus selects a level corresponding to the exercise capacity of the user in the selected exercise program. The apparatus provides the selected exercise program of the selected level to the user.

In operation 470, the apparatus receives a feedback on an exercise performance result from the user performing an exercise based on the exercise program selected in operation 460.

In operation 480, the apparatus adjusts the exercise program based on information associated with the feedback received in operation 470. The apparatus adjusts a level of exercise or changes the exercise program based on the information. Information on an exercise intensity or the exercise program adjusted in operation 480 is transmitted to operation 460 such that the apparatus selects the exercise program based on the information.

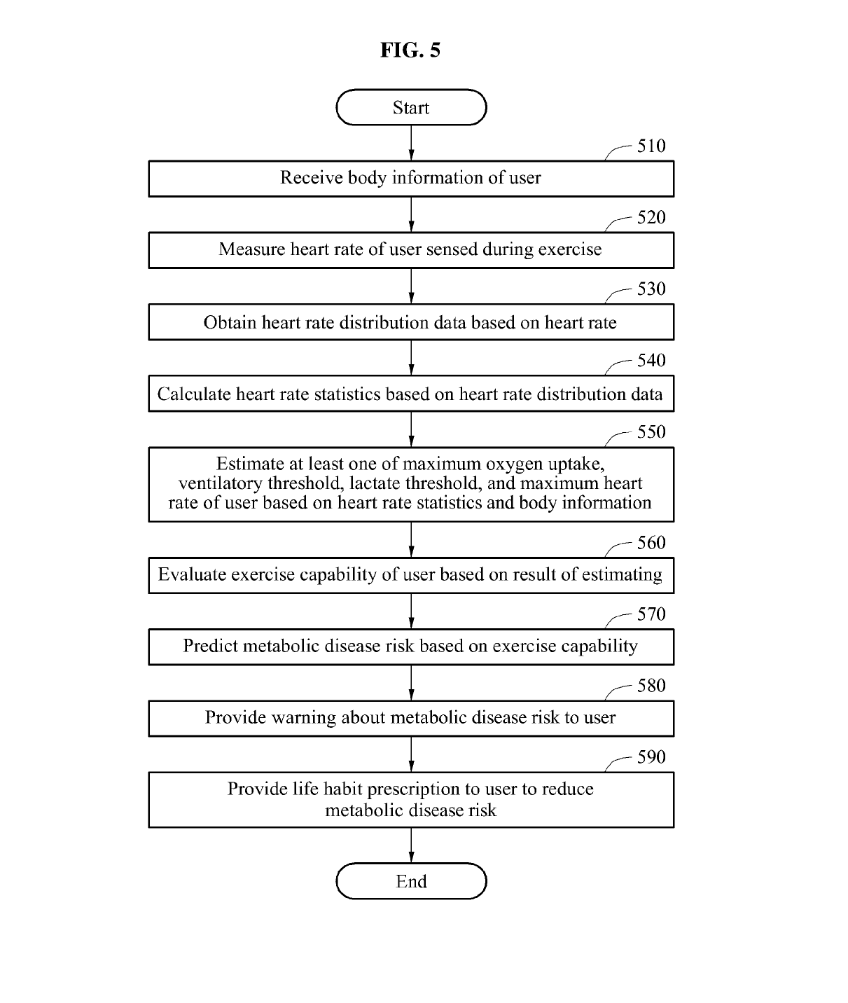

FIG. 5 is a flowchart illustrating still another example of a method of evaluating an exercise capacity. Referring to FIG. 5, in operation 510, an apparatus for evaluating exercise capacity receives body information of a user. The body information may be physiological information about the user's body. In this example, the body information may be directly input from the user. Alternatively, the body information may be a pre-stored value. The body information may be updated. The body information may include, for example, a gender, an age, a height, a weight, a waist-hip ratio (WHR), and a body mass index (BMI) of the user. The BMI may be obtained by dividing a weight by a square of a height. In this example, a unit of the weight is a kilogram (kg) and a unit of the square of the height is a square meter (m²).

In operation 520, the apparatus measures a heart rate of a user sensed while the user is performing an exercise in which an exercise load gradually increases.

In operation 530, the apparatus generates a heart rate distribution data based on the heart rate measured in operation 520. For example, the heart rate distribution data may be a heart rate distribution curve.

In operation 540, the apparatus calculates heart rate statistics based on the heart rate distribution data.

In operation 550, the apparatus estimates at least one of a maximum oxygen uptake, a ventilatory threshold, a lactate threshold, and a maximal heart rate of the user based on the heart rate statistics calculated in operation 540 and the body information received in operation 510.

The apparatus applies heart rate statistics X1, for example, a heart rate kurtosis, and body information X2, for example, a BMI, to a second estimation regression equation, for example, Y=α1×X1+α2×X2+β, to estimate at least one of the maximum oxygen uptake, the ventilatory threshold, the lactate threshold, and the maximal heart rate. Similarly, as in the first estimation regression equation, coefficients α1, α2, and β of the second estimation regression equation may be differently determined based on a metabolic index Y to be estimated, the heart rate statistics X1, and the body information X2.

Also, the apparatus applies heart rate statistics X1, such as a heart rate kurtosis, body information X2, such as an age, and body information X3, such as a BMI, to a third estimation regression equation, Y=α1×X1+α2×X2+α3×X3+β, to estimate a metabolic index of the user. Coefficients α1, α2, α3, and β of the third estimation regression equation may be differently determined based on the metabolic index Y to be estimated, and parameters, for example, the body information X2 and the body information X3.

In operation 560, the apparatus evaluates the exercise capacity of the user based on a result of the estimating performed in operation 550.

In operation 570, the apparatus predicts a metabolic disease risk based on the exercise capacity evaluated in operation 560. In operation 570, the apparatus calculates a healthiness grade of the user based on the exercise capacity of the user, and estimates a risk of death based on the healthiness grade.

In operation 580, the apparatus provides a warning, if any, about a metabolic disease risk to the user. For example, the apparatus may provide an auditory warning or display a written expression on a screen that states, “You are currently at the risk of metabolic disease.”

In operation 590, the apparatus provides a lifestyle prescription to the user to reduce the metabolic disease risk. In this example, the lifestyle prescription is understood as including, for example, an exercise prescription and/or a nutritional prescription. The lifestyle prescription may include recommendations regarding life habits.

Additionally, the apparatus provides information on an exercise suitable for a physical fitness level of the user by comparing the exercise capacity of the user to a standard exercise capacity of a person with the age and gender of the user based on the exercise capacity evaluated in operation 560.

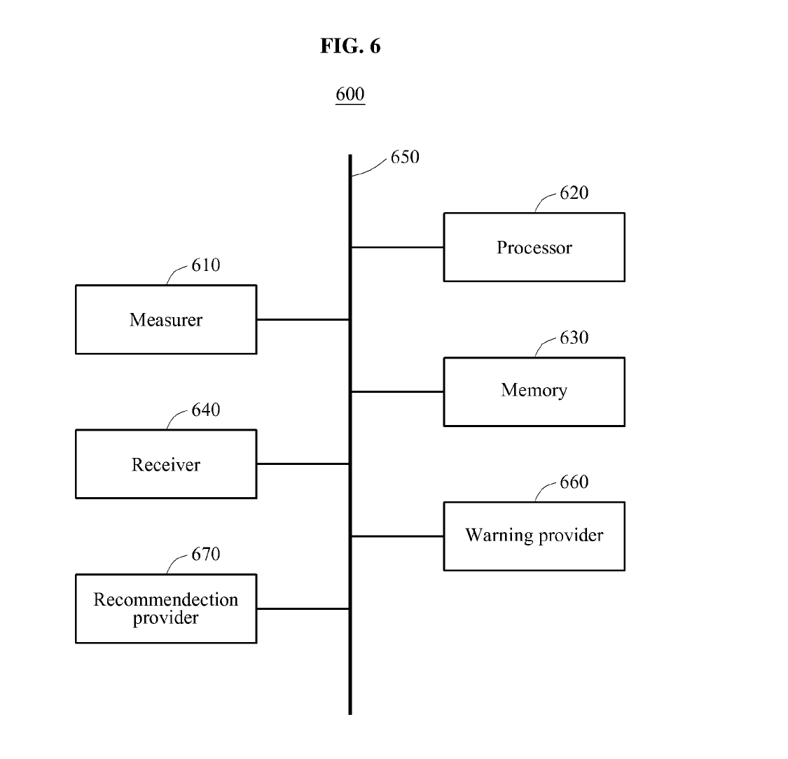

FIG. 6 is a block diagram illustrating an example of an apparatus 600 for evaluating exercise capacity.

Referring to FIG. 6, the apparatus 600 includes a measurer 610, a processor 620, a memory 630, and a receiver 640. The apparatus 600 may further include a warning provider 660 and a recommendation provider 670.

The measurer 610, the processor 620, the memory 630, the receiver 640, the warning provider 660, and the recommendation provider 670 communicate with one another through a bus 650.

The measurer 610 measures a heart rate of a user. The measurer 610 measures a heart rate sensed while the user performs an exercise in which an exercise load increases. The measurer 610 may include a heart rate sensor, such as a photoplethysmogram sensor.

The processor 620 calculates heart rate statistics from a heart rate distribution data generated based on the heart rate measured by the measurer 610, and evaluates an exercise capacity of the user based on the heart rate statistics. The processor 620 calculates at least one of skewness, kurtosis, and quantile of the heart rate based on the heart rate distribution data.

The processor 620 estimates at least one of a maximum oxygen uptake, a ventilator threshold, a lactate threshold, and a maximal heart rate of the user by applying an estimation regression equation to the heart rate distribution data. The processor 620 evaluates the exercise capacity of the user based on a result of the estimating.

The memory 630 stores at least one of the heart rate received from the measurer 610, the heart rate distribution data, and the heart rate statistics received from the processor 620.

The receiver 640 receives body information of the user from the user. The receiver 640 may include a computer processing unit configured to control the receiving of the body information and to store the body information in the memory 630. The receiver 640 may, for example, include a computer processing unit that causes a display screen or a touch screen to display a user interface to receive the body information from the user.

The processor 620 evaluates the exercise capacity of the user based on the heart rate statistics and the body information of the user. The processor 620 may include a computer processing unit, which is a hardware component, to evaluate the exercise capacity of the user.

The warning provider 660 predicts a metabolic disease risk of the user based on the exercise capacity evaluated by the processor 620, and provides a warning about a metabolic disease risk to the user. For example, the warning provider 660 calculates a healthiness grade of the user based on the exercise capacity of the user, predicts a risk of death due to a metabolic disease based on the healthiness grade of the user, and provides a warning about the risk of death to the user. The warning provider 660 may include a computer processing unit configured to predict the metabolic disease risk based on information stored in the memory 630 and the exercise capacity evaluated by the processor 620.

The recommendation provider 670 provides a lifestyle prescription to the user to reduce the metabolic disease risk. The lifestyle prescription may include an advice about a life habit, a recommendation for a lifestyle change, or a nutritional prescription about, for example, an exercise prescription combined with a diet change. The recommendation provider 670 may include a computer processing unit configured to display a user interface on a screen or direct other output devices, such as a speaker, to provide the lifestyle prescription to the user.

The processor 620 performs at least one of the methods described with reference to FIGS. 1 through 5. The processor 620 executes a program and controls the apparatus 600. A code of the program executed by the processor 620 is stored in the memory 630. The apparatus 600 is connected to an external apparatus, for example, a personal computer and a network through an input and output device (not shown) and performs a data exchange.

The examples of methods described with reference to FIGS. 1 through 6 may be implemented in a form of an application executed in a computer processor of a tablet computer, a smartphone, or a wearable device, or may be implemented in a form of a chip embedded in the smartphone or the wearable device.

The apparatuses, units, modules, devices, memories, measurers, receivers, providers, warning providers, and other components described with reference to or illustrated in FIG. 6 that perform the operations described herein with respect to FIGS. 2, 4 and 5 are implemented by hardware components. Examples of hardware components include controllers, sensors, generators, drivers, memories, comparators, arithmetic logic units, adders, subtractors, multipliers, dividers, integrators, and any other electronic components known to one of ordinary skill in the art. In one example, the hardware components are implemented by computing hardware, for example, by one or more processors or computers. A processor or computer is implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices known to one of ordinary skill in the art that is capable of responding to and executing instructions in a defined manner to achieve a desired result. In one example, a processor or computer includes, or is connected to, one or more memories storing instructions or software that are executed by the processor or computer. Hardware components implemented by a processor or computer execute instructions or software, such as an operating system (OS) and one or more software applications that run on the OS, to perform the operations described herein with respect to FIGS. 2, 4 and 5. The hardware components also access, manipulate, process, create, and store data in response to execution of the instructions or software. For simplicity, the singular term “processor” or “computer” may be used in the description of the examples described herein, but in other examples multiple processors or computers are used, or a processor or computer includes multiple processing elements, or multiple types of processing elements, or both. In one example, a hardware component includes multiple processors, and in another example, a hardware component includes a processor and a controller. A hardware component has any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 1, 4 and 5 are performed by a processor or a computer as described above executing instructions or software to perform the operations described herein.

Instructions or software to control a processor or computer to implement the hardware components and perform the methods as described above are written as computer programs, code segments, instructions or any combination thereof, for individually or collectively instructing or configuring the processor or computer to operate as a machine or special-purpose computer to perform the operations performed by the hardware components and the methods as described above. In one example, the instructions or software include machine code that is directly executed by the processor or computer, such as machine code produced by a compiler. In another example, the instructions or software include higher-level code that is executed by the processor or computer using an interpreter. Programmers of ordinary skill in the art can readily write the instructions or software based on the block diagrams and the flow charts illustrated in the drawings and the corresponding descriptions in the specification, which disclose algorithms for performing the operations performed by the hardware components and the methods as described above.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of evaluating exercise capacity, the method comprising:
obtaining heart rate distribution data based on measured heart rate of a user;
calculating heart rate statistics based on the heart rate distribution data; and
evaluating an exercise capacity of the user based on the heart rate statistics.

2. The method of claim 1, wherein the calculating of the heart rate statistics comprises:
calculating at least one of skewness, kurtosis, and quantile of the heart rate distribution data.

3. The method of claim 1, wherein the evaluating of the exercise capacity comprises:
estimating at least one of a maximum oxygen uptake, a ventilatory threshold, a lactate threshold, and a maximal heart rate based on the heart rate statistics; and
evaluating the exercise capacity of the user based on a result of the estimating.

4. The method of claim 3, wherein the estimating of the exercise capacity comprises:
applying the heart rate statistics to an estimation regression equation to estimate at least one of the maximum oxygen uptake, the ventilatory threshold, the lactate threshold, and the maximal heart rate of the user.

5. The method of claim 1, further comprising:
receiving body information of the user,
wherein the evaluating of the exercise capacity comprises evaluating the exercise capacity based on the body information and the heart rate statistics.

6. The method of claim 5, wherein the body information of the user comprises at least one of a gender, an age, a height, a weight, a waist-hip ratio (WHR), and a body mass index (BMI) of the user.

7. The method of claim 1, further comprising:
measuring the heart rate.

8. The method of claim 7, wherein the measuring comprises:
measuring a heart rate of the user while the user is performing an exercise in which a workload increases.

9. The method of claim 1, further comprising:
selecting an exercise program based on the exercise capacity of the user from pre-stored exercise programs;
receiving a feedback on an exercise performance result from the user performing an exercise based on the selected exercise program; and
adjusting the exercise program based on information associated with the received feedback.

10. The method of claim 1, further comprising:
comparing the exercise capacity of the user to a standard exercise capacity based on body information of the user; and
providing information on an exercise based on a physical fitness level of the user based on a result of the comparing.

11. The method of claim 1, further comprising:
predicting a metabolic disease risk of the user based on the exercise capacity; and
providing a warning about the metabolic disease risk.

12. The method of claim 11, wherein the predicting comprises:
calculating a healthiness grade of the user based on the exercise capacity of the user; and
estimating a risk of death based on the healthiness grade of the user.

13. The method of claim 11, further comprising:
providing a lifestyle prescription to reduce the metabolic disease risk of the user.

14. A non-transitory computer readable medium storing instructions that cause a processor to perform the method of claim 1.

15. An apparatus for evaluating exercise capacity, the apparatus comprising:
a measurer configured to measure a heart rate of a user; and
a processor configured to calculate heart rate statistics from heart rate distribution data obtained based on the measured heart rate and evaluate an exercise capacity of the user based on the heart rate statistics.

16. The apparatus of claim 15, further comprising:
a memory configured to store at least one of the heart rate, the heart rate distribution data, and the heart rate statistics.

17. The apparatus of claim 15, wherein the processor is configured to calculate at least one of skewness, kurtosis, and quantile of the heart rate distribution data.

18. The apparatus of claim 15, wherein the processor is configured to estimate at least one of a maximum oxygen uptake, a ventilatory threshold, a lactate threshold, and a maximal heart rate by applying the heart rate statistics to an estimation regression equation, and evaluate the exercise capacity of the user based on a result of the estimating.

19. The apparatus of claim 15, further comprising:
a receiver configured to receive body information of the user,
wherein the processor is configured to evaluate the exercise capacity of the user based on the body information and the heart rate statistics.

20. The apparatus of claim 15, wherein the measurer is configured to measure a heart rate of the user while the user is performing an exercise in which a workload increases.

21. An apparatus for evaluating exercise capacity, the apparatus comprising:
a processor configured to obtain heart rate distribution data based on a heart rate of a person measured while performing a predetermined exercise regime, and evaluate an exercise capacity of the person based on a calculation performed on the heart rate distribution data,
wherein the apparatus is a wearable device or a mobile device.

22. The apparatus of claim 21, further comprising:
a heart rate sensor configured to measure the heart rate of the person.

23. The apparatus of claim 21, wherein the calculation performed on the heart rate distribution data comprises determining at least one of a maximum oxygen uptake, a ventilatory threshold, a lactate threshold, and a maximal heart rate.
